(19) United States
(12) Patent Application Publication
(10) Pub. No.: US 2021/0187165 A1
(43) Pub. Date: Jun. 24, 2021

(54) Collagen Tissue Modification

(71) Applicant: The Trustees of Columbia University in the City of New York, New York, NY (US)

(72) Inventors: Sinisa VUKELIC, New York, NY (US); Gerard A. ATESHIAN, New York, NY (US); Chao WANG, New York, NY (US); Krista M. DURNEY, Upton, NY (US)

(73) Assignee: The Trustees of Columbia University in the City of New York, New York, NY (US)

(21) Appl. No.: 17/122,392

(22) Filed: Dec. 15, 2020

Related U.S. Application Data
(63) Continuation of application No. PCT/US2019/040728, filed on Jul. 5, 2019.
(60) Provisional application No. 62/694,695, filed on Jul. 6, 2018.

(51) Int. Cl. A61L 27/36; A61L 27/24

(52) U.S. Cl. CPC A61L 27/3687; A61L 27/24; A61L 2430/40; A61L 27/3662; A61L 2430/06; A61L 27/3654

(57) ABSTRACT

Treatment systems and methods are disclosed that may be used to strengthen cartilage. The disclosed subject matter includes description of this advantage and associated experimental results. The systems and methods employ ultrafast laser-based treatment to induce crosslinks into the collagen network of the tissue media without the addition of a chemical agent. The system and related methods may also be used for other purposes discussed herein.

### CROSS-REFERENCE TO RELATED APPLICATIONS

This application is a continuation of international application PCT/US2019/040728, filed on Jul. 5, 2019, which claims priority to and the benefit of U.S. Provisional Patent Application No. 62/694,695 filed Jul. 6, 2018, each of which is incorporated herein by reference in its entirety.

### GOVERNMENT SUPPORT

This invention was made with government support under AR073289 awarded by the National Institutes of Health. The government has certain rights in the invention.

### BACKGROUND

More than 27 million Americans suffer from degenerative diseases of articular cartilage, such as osteoarthritis (OA). While the expected lifetime of the load-bearing cartilage tissue should coincide with the lifespan of an individual, it has a limited ability to self-repair and the damage to the tissue can accumulate severely. The primary function of articular cartilage is to transmit loads across the joint surfaces while simultaneously minimizing friction and wear. Progressive OA results in debilitating and painful loss of joint function. Most therapies, such as highly invasive partial and total joint replacement surgeries are performed at the late stage of the disease.

Osteoarthritis (OA) affects millions of Americans. OA is a progressive, complex, multi-tissue joint disease with degenerative changes in the articular cartilage and subchondral bone, with a long asymptomatic early development and debilitating late stages. Late-stage treatment options are limited to major interventions, including joint replacement.

### SUMMARY

Introducing treatment options to earlier stages of OA may be used to slow down disease progression and thus significantly improve patient outcomes. While OA is understood today to be a disease of a joint as an organ, with bone, cartilage, and synovial fluid involved the pathological progression may occur due to the initial disturbance of the extracellular matrix (ECM) homeostasis in articular cartilage. Ultrafast laser-based treatment has the ability to induce crosslinks into the collagen network without the addition of a chemical agent to avoid damaging effects of optical breakdown and ablation. Data show that laser-induced crosslinks increase compressive stiffness and wear resistance, which may slow down progression of OA.

While OA is viewed today as a disease of the joint as an organ, with inflammation, injury, and changes in bone, articular cartilage, and synovial fluid as potential driving forces, the degradation of the ECM components of cartilage is key to the progression of the disease. Adult articular cartilage is an avascular connective tissue composed of ECM and chondrocytes, with nutrition and metabolites exchanged with synovial fluid by diffusion. The key structural components of cartilage ECM are collagens (collagen) and proteoglycans; chondrocytes synthesize and degrade all components of the ECM. While highly hydrophilic proteoglycans provide hydrostatic pressure, the interwoven crosslinked collagen fibril network provides cartilage tensile strength and counteracts the swelling pressure of proteoglycans. Healthy cartilage functions as an effective shock absorbing tissue with minimal friction and wear for smooth joint movement. The homeostasis of ECM in cartilage is dependent on the dynamic signaling by anabolic and catabolic factors (cytokines and growth factors). Whether the initiating factors of OA are acutely mechanical (trauma) or gradually developing with age, an imbalance in ECM homeostasis is a key pathogenic pathway of OA. The disruption of collagens network leads to its inability to withstand the swelling pressure of proteoglycans, resulting in increased water content. Along with swelling, increased proteolytic activity shifts the ECM homeostasis towards degradation of matrix components, progressively leading to cartilage degeneration and loss of function.

### BRIEF DESCRIPTION OF DRAWINGS

FIG. 1A shows laser scan of test sample.

FIG. 1B shows photographs of the test samples of FIG. 1A.

FIG. 2 shows a human OA distal femur.

FIG. 3A shows a human femoral condyle (human femoral condyle), with evidence of delamination.

FIG. 3B shows a human femoral condyle (human femoral condyle) showing missing surface zone.

FIG. 3C shows a delaminated bovine cartilage strip.

FIG. 3D shows an OA plug.

FIG. 4 shows a matrix showing control bovine cartilage explant.

FIG. 5 shows a treatment process for the treatment of cartilage.

FIGS. 6A through 6D illustrate laser-tissue interaction mechanism in collagen tissues such as cartilage.

FIG. 7 shows slides of representative bovine cartilage plugs after wear testing.

FIGS. 8A and 8B show stiffening of articular cartilage with laser treatment.

FIG. 9 shows a relationship between friction coefficient and time.

FIGS. 10A through 10D show live/dead staining of immature bovine cartilage explants.

FIG. 11 illustrates an operating range for laser parameters.

FIG. 12 shows a subcutaneous dorsum pouch.

FIG. 13 shows a schematic diagram of the fiber optic probe and its application.

FIG. 14A show laser setups.

FIGS. 14B through 14C show laser beam profiles.

FIG. 15 shows two friction testing devices.

### DETAILED DESCRIPTION

The onset of OA is characterized by changes to the structure, though not necessarily the content, of collagen matrix in articular cartilage. Crosslinks stabilize the collagen fiber network of cartilage, and their disruption leads to the loss of tensile strength and structural integrity. In cartilage, hydroxylysyl pyridinoline is the major mature crosslinks and pentosidine is the senescent one. Lysyl oxidase mediates covalent crosslinking of collagen fibrils by oxidizing hydroxylysine residues to hydroxylysyl aldehydes which then, through several reactions, lead first to immature pentosidine, then to stable hydroxylysyl pyridinoline crosslinks. The contribution of crosslinking to tensile strength and stability of cartilage collagen matrix has been extensively demonstrated in tissue engineering studies. In animal models of OA, crosslinking loss determined the irreversibility of cartilage ECM degeneration, irrespective of the level of proteoglycans loss. In patients, increased levels of hydroxylysyl pyridinoline and pentosidine in urine correlated with the severity of OA and other degenerative joint diseases.

Treatment may employ the femtosecond laser for selective and localized alteration of the biomechanical properties of articular cartilage such that ionization of target molecules within cartilage is achieved while avoiding optical breakdown of the tissue. The system and method do not rely either on thermal ablation or optical breakdown of cartilage material. By imparting low-density plasma, an ionization field is generated within the media of the cartilage without any damaging thermoacoustic and shock waves. Imparting low-density plasma to cartilage, which is an example of hydrated collagenous tissues, generates low-density plasma and produces reactive oxygen species by breaking down water. These species react with surrounding proteins resulting in crosslink formation in cartilage and give rise to spatially resolved modification of mechanical properties.

The envelope of effective and safe laser operating parameters (energy and numerical apertures) were optimized using laboratory and clinical tests, for example, using devitalized immature bovine and mature human cartilage from OA joints. Optimization targets include effectiveness and safety of treatment which may be measured from evaluation of compressive modulus and wear resistance. Data confirm enhancement of compressive modulus and wear resistance as described herein.

The above-determined optimum range of operating parameters may be narrowed by testing (a) short-term and (b) long-term viability of laser-treated live bovine and human (male & female) explants against untreated controls, using in vitro culture up to 4 weeks. Such tests identify the range of parameters that maintain chondrocyte viability 24 hours post laser treatment. Such tests will indirectly determine whether short-lived bursts of reactive oxygen species (ROS) have a long-term influence on tissue viability and health. Preliminary data confirm short-term viability and viability at 2 weeks as described below.

Live human OA cartilage explants may be treated and verified to exhibit the same viability and health as untreated controls when implanted for up to 8 weeks in the back of nude mice. Such tests may provide an in vivo assessment of safety in response to short-lived bursts of ROS.

The limited area of laser treatment was found not to introduce a deleterious amount of matrix heterogeneity on the articular cartilage surface indicated by wear tests on 2 pairs of immature bovine cartilage discs laser-treated at their center indicate the contact area migrated past the treated region. In the untreated control group, both samples delaminated before completion of 12 h of sliding against a glass lens; in the lased group, both samples showed no wear after 12 h.

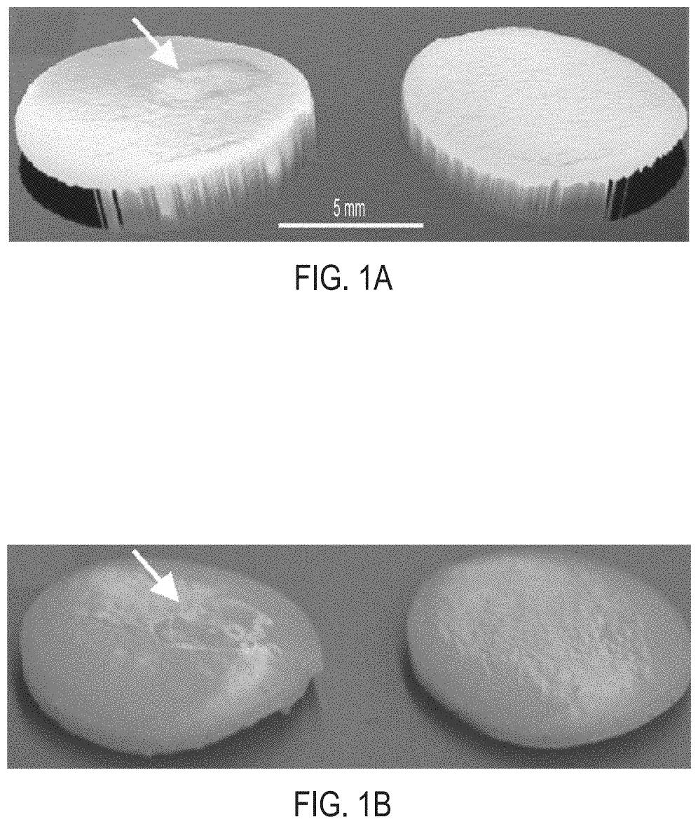

FIG. 1B shows photographs and FIG. 1A shows laser surface scans of 010 mm×1.2 mm bovine cartilage plugs after sliding for 12 h, 4 mm travel at 1 mm/s, against a spherical glass lens (R12.7 mm) under 4.45 N load. Arrows indicate blister due to delamination wear in control sample (left). Treated sample (right) was lased over a 3 mm×3 mm central patch and showed no damage.

Presented data includes histological and visual evidence that the human samples used in the studies are fibrillated and come from human joints whose overall Outerbridge score is 3. Experiments on these fibrillated human cartilage samples showed that laser treatment can enhance their compressive modulus more than three-fold compared to sham-treated controls.

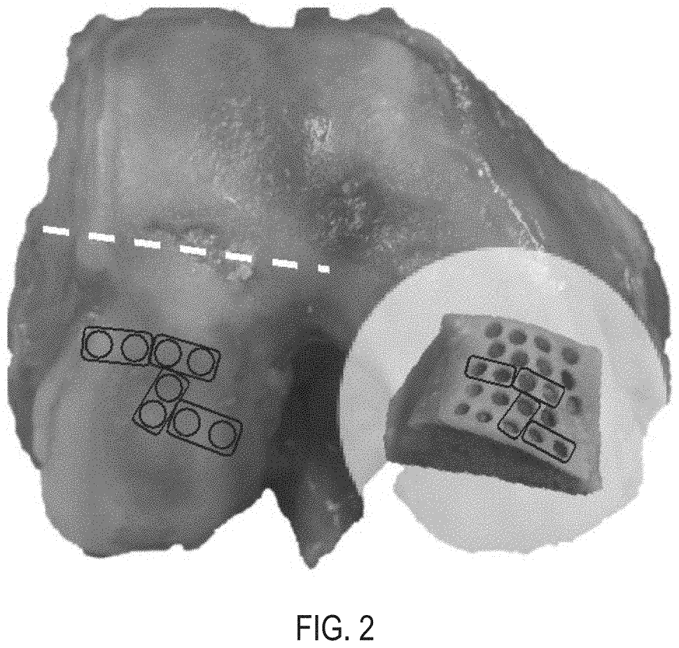

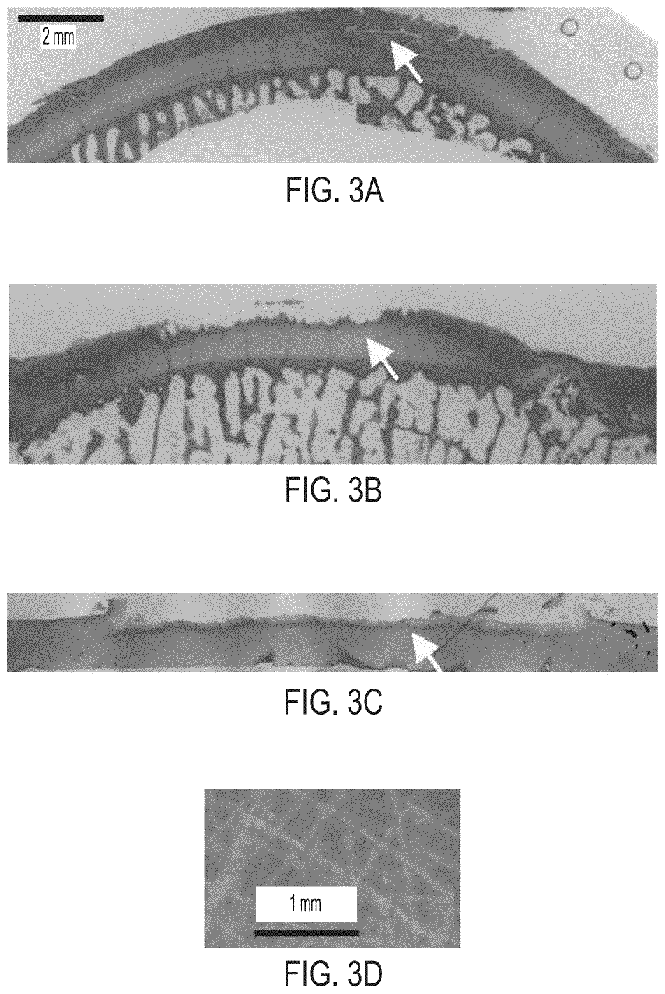

FIG. 2 shows a human OA distal femur with overall Outerbridge score 3. Dashed line represents the histology section shown in FIG. 3B. Circles on lateral condyle (left) are sites of tissue harvest for laser treatment study, rectangles show paired samples. Inset (bottom right) shows harvested sites of samples used for results reported herein.

FIG. 3A shows a human femoral condyle, with evidence of delamination (arrow). FIG. 3B shows a human femoral condyle (human femoral condyle) showing missing surface zone, with typical fibrillation. FIG. 3C shows a delaminated bovine cartilage strip that shows fibrillated surface (arrow). FIG. 3D shows a typical OA plug from, graded OS2, test results.

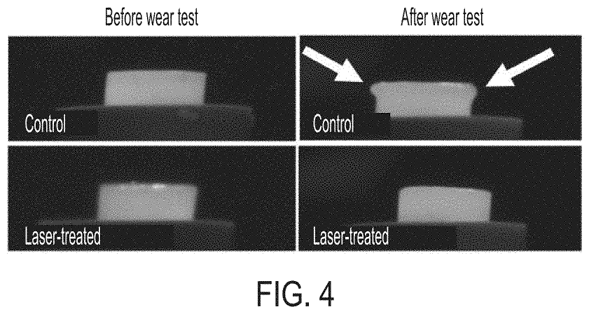

FIG. 4 shows a matrix showing control bovine cartilage explant (Ø3 mm×1 mm), before and after wear test, demonstrates greater visual evidence of surface damage (arrows) compared to laser-treated explant (side view).

Results show that delamination of bovine cartilage under migrating contact of glass against large cartilage strips is observed, with initial evidence in the form of a delaminated blister, leading to complete removal of the superficial zone (blister surface) upon further testing, with the appearance of surface fibrillation. Presently presented are histological evidence from human OA knee joints with some regions clearly showing the initiation of delamination and others exhibiting a fibrillated surface with substantially similar morphology to bovine migrating contact area test results. Therefore, wear of human cartilage appears similar to the in vitro wear model.

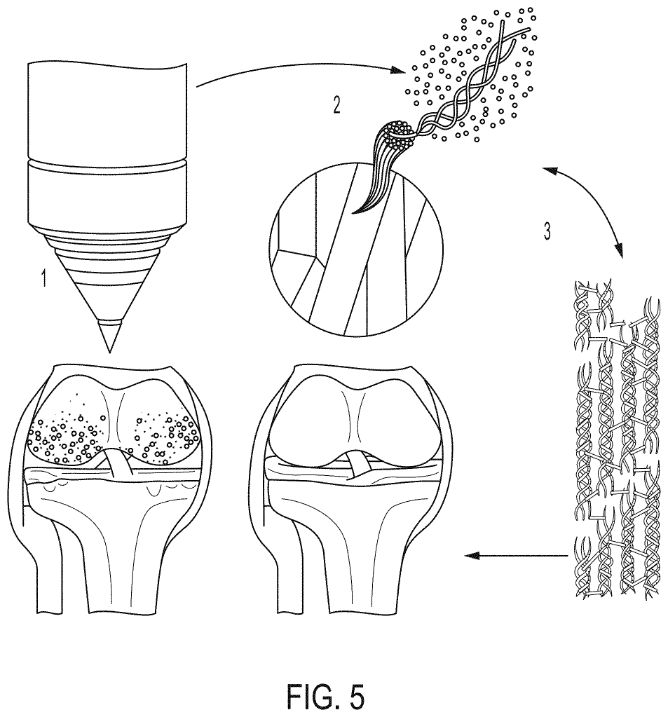

FIG. 5 Shows a treatment process. (1) A femtosecond laser irradiates articular cartilage creating an ionization field; (2) ionization generates reactive oxygen species which interact with collagen fibrils in the ECM; (3) biochemical reactions with ROS result in human femoral condyle formation, which enhance cartilage mechanical properties, potentially slowing down OA progression.

Current use of lasers in OA treatment is divided into two distinct fields, disease management and techniques related to laser-assisted tissue ablation such as debridement and abrasion, used in the arthroscopic treatment of OA and cartilage lesions. In the latter, the laser is used as a thermal source to perform chondroplasty, aiming to smooth the fibrillated articular cartilage surface. For some practitioners lasers are seen as a more effective replacement to mechanical chondroplasty. However, commonly used lasers such as excimer and solid state lasers cause significant thermal injury to the joint that can lead to avascular necrosis of adjacent subchondral bone, affecting the viability of the remaining articular cartilage. In this application, the proposed treatment modality utilizes an ultrafast oscillator, which has extremely low pulse energy (˜1.2 nJ), and as such does not inflict thermal damage, as demonstrated in previous work on porcine cornea. Smoothing of osteoarthritic cartilage has been proposed to be beneficial because it can reduce friction and further wear. However, shaving off fibrillated layers of cartilage may also remove biomechanically protective regions. Furthermore, shaving treatment does not improve the biomechanical properties of OA-afflicted cartilage and the resulting pain relief may only be temporary. In particular, it has been shown that excimer laser-treated articular cartilage in rabbits has inferior structural integrity when compared against normal tissue, despite its cartilage-like morphological appearance.

For a long time, arthroscopic debridement represented the standard treatment for early OA. Starting from the early 2000s, rigorous outcome studies began to show that debridement was no more effective than a sham control for the purpose of relieving pain or improving function. Other modalities, such as viscosupplementation with injectable hyaluronic acid, have also produced mixed results, with no significant difference against placebo. When cartilage lesions are deep or subchondral bone is exposed, microfracture techniques have been successfully used to induce fibrocartilaginous tissue growth, though this approach does not actually repair the existing tissue. Therefore, when a surgeon detects local cartilage fibrillation or softening during arthroscopy, no reliable modalities exist today to provide localized repair of damaged extant tissue. Non-ablative laser treatment method induces a photochemical reaction in cartilage that results in production of crosslinks. Data show that these newly induced crosslinks enhance the mechanical and wear properties of healthy bovine and osteoarthritic human cartilage.

OA is often described as a natural process of wear and tear associated with aging, or an initiating traumatic event. The cartilage mechanics literature has mostly focused on examining the friction coefficient μ as a surrogate for understanding wear and tear in cartilage. The friction coefficient μ is the ratio of the tangential force to the normal contact force acting across the bearing surfaces. The friction coefficient of articular cartilage is not constant. The lowest reported value of μ for cartilage against glass is typically μ≈0.002, which is exceptionally low. However, μ may rise over time, depending on loading conditions, to achieve values as high as μ≈0.15 against glass, or even μ≈0.5 against stainless steel. These values are detrimental to cartilage. When cartilage is loaded against cartilage, the friction coefficient remains constant for sustained durations; for human cartilage, it is typically μ≈0.020 in synovial fluid (SF) and μ≈0.025 in saline. Implicitly, a low value of μ has been assumed to produce low wear while an elevated value could lead to significant wear. Despite the prominence of this hypothesized mechanism, it is only recently that cartilage wear studies have started to be performed under controlled conditions, most notably by investigating PRG4 knockout mice, since PRG4/lubricin has been shown to reduce the friction coefficient of cartilage in vitro and prevent degeneration in vivo. Few studies have investigated the ability of living cartilage to repair, following episodes of tissue wear. Wear is a generally complex phenomenon that may manifest itself in different ways. In the engineering tribology literature (tribology=science of lubrication and wear), a broad range of wear mechanisms are reported, many of which are mostly applicable to metals and other artificial surfaces. However, some of these mechanisms may also be candidates for wear of biological tissues, such as abrasive wear, which removes particulates of matter from the bearing surfaces; third-body wear, where particulate matter causes further abrasion of the bearing surfaces; fatigue wear with delamination, where the load-bearing material fails below the surface due to fatigue and the failure propagates until a lamina shears off; and chemical wear, where breakdown of the bearing material is initiated by chemical reactions, such as proteolysis in biological tissues. It is unclear which of these mechanisms prevail in articular cartilage, and under what conditions. It is believed that the prior applicant studies show that the primary wear mechanism in cartilage is fatigue wear with delamination of the surface zone. Delamination is a clinically recognized symptom of OA. Laser treatment may strengthen the sub-surface region of cartilage to reduce the propensity for wear progression by delamination.

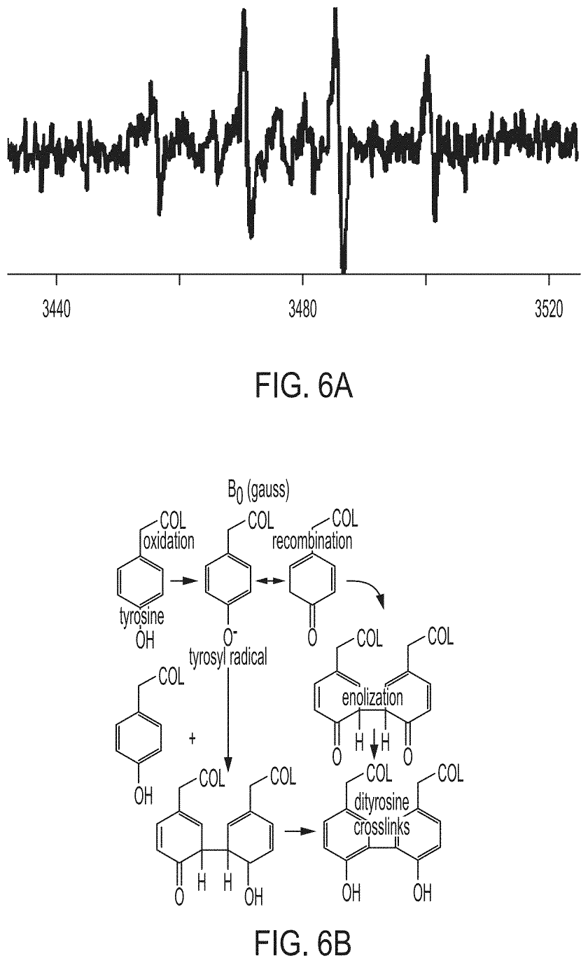

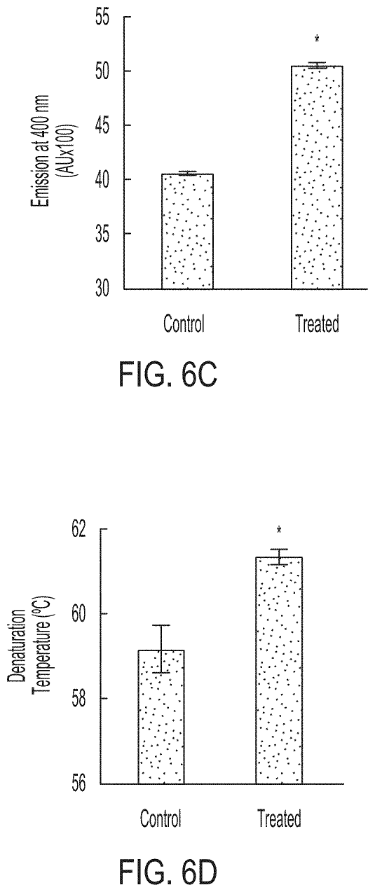

FIGS. 6A through 6D show laser-tissue interaction mechanism in cornea. FIG. 6A shows electron Paramagnetic Resonance spectrum: Spin-trap reagent 5,5-dimethy-1-pyrroline-N-oxide solved in Dulbecco's phosphate-buffered saline has trapped OH* and O2-, created after the solution was ionized with femtosecond oscillator. Fig. B shows oxidative modification of tyrosine: Specific oxidative amino acid modification associates with abstraction of the phenolic hydrogen atom from tyrosine residues—tyrosyl radical. The tyrosyl radical is combined with another one to generate a stable, covalent, carbon-carbon bond forming 1,3-dityrosine. FIG. 6C shows fluorescence spectrum of laser-treated and control samples of 5 mM tyrosine solution in pH 10 Tris buffer measured at 400 nm emission and 325 nm excitation. FIG. 6D shows Differential Scanning calorimetry: The thermal denaturation temperature of the treated samples is −2° C. higher than that of untreated samples. *p<0.05.

The disclosed mode of laser-tissue interaction avoids both thermal ablation and optical breakdown to enhance mechanical properties of OA affected articular cartilage. The method relies on constraining the laser treatment regime such that optical breakdown never occurs. Disclosed subject matter demonstrates the effectiveness. A treatment process is outlined in FIG. 5. When ultrashort pulses carrying nano-joule (nJ) energy are relatively loosely focused onto cartilage media, the interaction results in the formation of a low-density plasma within the focal volume and its immediate vicinity. low-density plasma regime has been previously observed and reported, with second harmonic imaging showing that it alters the signal from cartilage which is a collagen rich tissue. The phenomenon is generally regarded as an undesired side-effect occasionally present in multiphoton imaging. In the embodiments, an LPD is used to generate an ionization field in biological media, without producing tissue-damaging thermoacoustic and shock waves. The ionization field locally ionizes and dissociates interstitial water, creating reactive oxygen species which interact with surrounding proteins to form human femoral condyle, giving rise to spatially resolved alterations in mechanical properties.

ROS-induced formation of intra- and inter-molecular covalent bonds between collagen fibrils has been previously observed in published work by an inventor or inventors of the present application. Independently, the production of ROS as a byproduct of plasma generation in aqueous media has been the subject of considerable interest on its own. Formation of free radicals through 2-photon ionization and dissociation of water molecules has initially been achieved by irradiation with high-power UV picosecond lasers. Advances in femtosecond lasers have enabled phasing to multiphoton ionization. In aqueous environments, laser-induced ionization and dissociation occur as a cascade of reactions that can be classified as primary, secondary and tertiary. Primary reactions include the formation of solvated electrons and the cation radical of water H2O+. The latter is unstable and reacts with a water molecule producing a hydrogen ion H3O+, and hydroxyl radical OH*. Concurrently, dissociation of the excited water molecule occurs, H2O*→H+OH*, providing another OH*. Primary reactions are followed by secondary and tertiary reactions in which the formation of H, O2-, OH—, H2, H2O2, HO2 and other species occur.

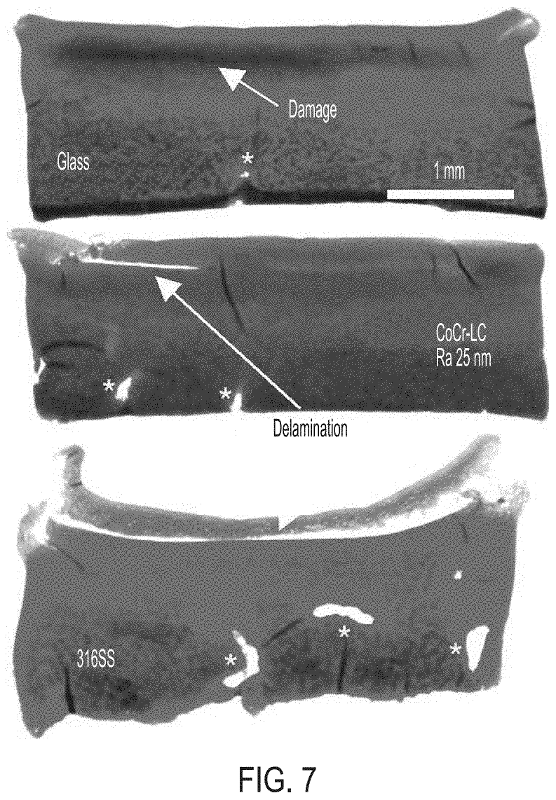

FIG. 7 shows slides of representative bovine cartilage plugs after wear testing against various materials (Safranin-O staining for GAG), showing damage and delamination. * denotes cartilage canals.

Laser intensities employed in studies focused on ROS generation have been well above the irradiance threshold for femtosecond breakdown in aqueous and ocular media (˜10¹³ W/cm²), a level at which density of photoionization-formed free electrons reach a critical value, resulting in formation of a dense, optically opaque plasma. However, since the number of free electrons produced during a single pulse is a function of irradiance, one could focus pulses generated by a femtosecond oscillator on biological media such that the density of the laser-generated free electrons is well below the critical value needed for the formation of dense plasma, but significant enough for the generation of low-density plasma. In such a scenario, although the femtosecond irradiation is below the energy level required for optical breakdown, ionization of atoms within the focal volume is possible because the ionization probability has a number of resonance maxima due to intermediate transition of the atom to an excited state. In the vicinity of such a maximum, the ionization cross-section increases by several orders of magnitude, enabling ionization even if the frequency of the incoming electromagnetic wave is lower than the ionization potential. Multiple photons interact simultaneously with a bound electron to overcome the band gap, and produce an electron-hole pair. Under these conditions, ionization of aqueous media may occur and low-density plasma produces ROS in the aqueous solutions as confirmed by the use of human femoral condyle spectroscopy to capture OH* in a aqueous solution irradiated by a femtosecond oscillator.

ROS initiate photo-oxidation of proteins, which results in the formation of chemical Crosslinks. All amino acids are susceptible to modification by *OH and *OH+O2-(+O2) radicals; however, tryptophan, tyrosine, histidine, and cysteine are particularly sensitive. Amino acids involved in human femoral condyle formation include histidine, hydroxylysine and tyrosine. Oxidative modification of tyrosine is characterized by abstraction of phenolic hydrogen atom from tyrosine residues. The tyrosyl radical is relatively long-lived and can react with another tyrosyl radical or tyrosine to form a stable, covalent carbon-carbon bond, resulting in the creation of 1,3-dityrosine. This formation is a product of protein oxidation which leads to intra- or intermolecular crosslinking. The reaction serves as a primer of pathways that lead to crosslinking of ECM upon irradiation with a femtosecond oscillator. This technology is highly novel and its successful application to collagenous tissues represents a unique contribution from this team of investigators.

Wear of cartilage against materials used in joint hemiarthroplasties was studied to observe that the native cartilage layer in a hemiarthroplasty undergoes accelerated cartilage wear. Immature bovine cartilage disks were tested in phosphate buffered saline containing protease inhibitors, in an unconfined compression configuration that accelerated loss of interstitial fluid pressurization, to replicate conditions believed to occur in hemiarthroplasties (on a much longer time scale). The prescribed contact stress was low (0.18 MPa) and reciprocal sliding (±5 mm at 1 mm/s) was performed for 4 h (N=1,440 cycles). Results showed that considerably more damage occurred in cartilage samples tested against 316SS stainless steel and cobalt-chromium alloy (CoCr-LC-Ra 25 nm) compared to glass. Cartilage damage occurred primarily in the form of delamination at the interface between the superficial tangential zone (SZ) and the transitional middle zone (MZ). Based on histology and low particulate volume, there was little evidence of abrasive wear at the articular surface. In the cartilage samples tested against 316SS and CoCr LC-Ra25 nm, the delamination was occult, becoming visible only on histological sections. However, evidence of sub-surface, pre-delamination damage was also found for glass. These results were confirmed in a more physiological migrating contact area (MCA) testing configuration that sustains interstitial fluid pressurization and low friction, sliding a glass lens (R12.7 mm) against immature bovine cartilage strips (30 mm×10 mm×1.2 mm, n=8) under a contact stress of 1.3 MPa for 8 h in protease inhibitors. Delamination occurred at the center of the strips (thus, no edge effects), manifested by a blister visible on 3D scans and photographs, as well as polarized light microscopy (PLM) of a cross-section through the blister, with evidence of fibrillation with further wear testing, when the SZ was removed. These results imply that cartilage damage from frictional loading occurs as a result of subsurface fatigue failure leading to the delamination. This delamination at the SZ-MZ interface is consistent with findings that this region exhibits the lowest shear modulus across the articular layer thickness, in bovine and human cartilage. Thus, it sees the greatest amount of reciprocal shear strain under frictional shear, causing fatigue failure.

The laser treatment protocol targets the subsurface region, located approximately within 300 μm of the articular surface. By strengthening this region with enhanced cross-linking, cartilage may demonstrate greater resistance to fatigue failure than untreated controls. Tests of bovine cartilage plugs in reciprocal sliding against glass, under a constant applied load for 4 hours represents an accelerated in vitro model of damage. Previous work has previously shown that no such damage occurs when loading healthy human shoulder joints (cartilage-against-cartilage) for 24 hours under physiological load magnitudes due to sustained interstitial fluid pressurization under those conditions and greater strength of healthy mature human tissue.

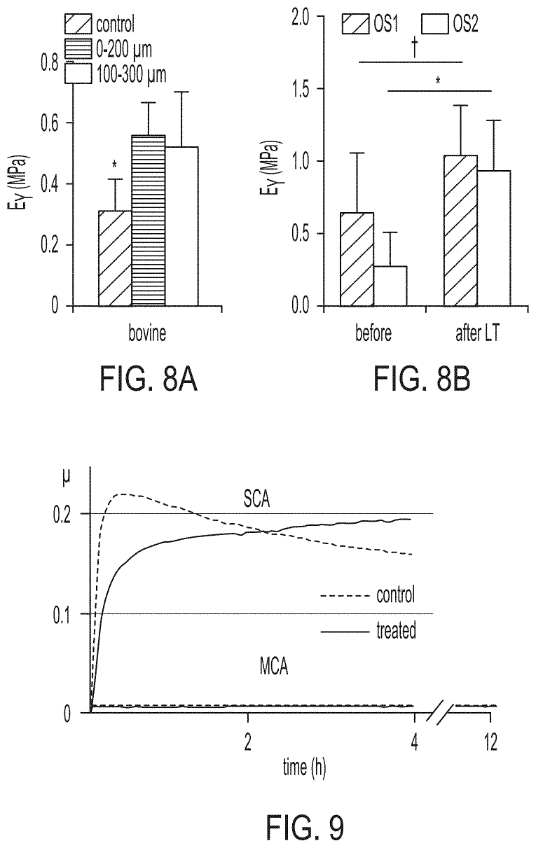

FIGS. 8A and 8B show stiffening of articular cartilage with laser treatment. FIG. 8A shows immature bovine treated from 0-200 μm and 100-300 μm from surface (PS1). FIG. 8B shows mature human OA cartilage with Outerbridge scores 1 and 2, treated from 0-200 μm from surface (PS2), *p<0.005, †p<0.05.

In PS1, devitalized immature bovine cartilage explants (03 mm×1 mm) were tested in a two-factorial experimental design: Factor 1 tested the effect of laser treatment dosage at a given energy level (0, 1 and 2 rasterized laser treatment passes of the explant surface, with 0 representing the untreated control group); factor 2 tested the effectiveness of the treatment with increasing depth from the articular surface (focusing the laser at depths of 0-200 μm, and 100-300 μm). This 3×2 design had n=6 explants tested in each cell (total of 36 explants). The principal outcome measure was the compressive equilibrium unconfined compression modulus Young's modulus. Results showed that 1 and 2 passes enhanced Young's modulus by nearly 100%, producing a significantly higher Young's modulus than the 0-pass control group (p<0.03). This enhancement was equally effective at the two ranges of depths from the articular surface (p=0.47). These results indicate that one pass is sufficient to saturate the cross-linking sites available for this modality and a doubling of the treatment dosage exhibited no mechanical evidence of harmful effects. Subsequent preliminary tests could be performed at the same energy level, using one pass.

In addition to enhancing the modulus Young's modulus, the wear resistance of control and laser-treated devitalized immature bovine explants, in a subset of samples from the study (n=4 per group). Explants were subjected to sliding against glass for 4 h, using the protocol described in an earlier study. For control samples, Young's modulus decreased significantly with wear testing, from 0.38±0.01 MPa down to 0.32±0.02 MPa (p<0.03), exhibiting clear visual evidence of damage. In contrast, laser-treated samples exhibited no significant difference in Young's modulus (p=0.13), going from 0.50±0.03 to 0.49±0.03 MPa before and after the wear test. These samples showed little visual evidence of damage. Bovine cartilage discs were tested under MCA, where laser treatment was only performed at the center region, against untreated controls, using the protocol described in an earlier study. Furthermore, the friction coefficient remains comparable in magnitude between control and laser-treated cartilage, both for MCA and for stationary contact area (SCA) as performed in PS1. These results suggest that the most important functional properties of cartilage (sustaining compressive load and producing low wear) are significantly enhanced by the disclosed laser treatment methodology, without detriment to the friction coefficient. While SCA exhibits much higher friction than MCA due to loss of fluid pressurization, eventually they both lead to similar delamination, justifying the use of the less time-consuming SCA protocol here.

FIG. 9 shows that friction coefficient μ remains low over MCA 12 h wear test (μ=0.008 damaged control, μ=0.005 undamaged treated). Typical results from SCA 4 h test show much higher, time-dependent μ for both control and treated.

In a third preliminary study (PS3), live immature laser-treated bovine cartilage explants (0 and 1 pass, from 100 to 300 μm, n=12 per group) and examined Young's modulus and cell viability (Live/Dead Assay Kit, Molecular Probes) 24 h after laser treatment. The compressive stiffness exhibited a similar increase with laser treatment (p<0.03), from Young's modulus=0.36±0.21 MPa (0 pass) to Young's modulus=0.58±0.21 MPa (1 pass). Confocal images of explants demonstrated no evidence of viability loss from laser treatment. For additional samples maintained for two weeks in culture after laser treatment, cell viability was similarly maintained when compared to untreated controls. These results demonstrate that laser treatment does not compromise cell viability for up to two weeks, while achieving equally effective enhancement of the tissue compressive modulus as with devitalized explants. These highly encouraging results may be examined more thoroughly in the studies proposed in this application, by culturing control and treated explants for up to 4 weeks post-treatment to confirm long-term cell viability and enhancement of other properties, such as wear resistance, without compromising tissue composition (collagen and proteoglycans). A subset of laser treatment parameters were identified that produce successful outcomes; these results are the evidence that cartilage properties may be enhanced with laser treatment.

The goal of this application is to introduce a practical treatment modality capable of stopping or slowing structural degeneration of articular cartilage in OA. Ultrafast lasers achieve this goal by inducing a photochemical effect rather than optical breakdown or photoablation.

Optical breakdown occurs if laser irradiance is approximately 10¹³ W/cm², characterized by formation of a shock wave that disrupts surrounding tissue by creating a cavitation bubble. Even if the transferred energy is below this optical breakdown level, the lasing energy may still create thermoacoustic waves. In this scenario, a considerable amount of heating occurs within and in the immediate vicinity of the focal volume, resulting in the formation of a transient bubble with a radius of 120-300 nm. The bubble disrupts the surrounding tissue and, if applied onto articular cartilage, would likely denature the ECM matrix, leading to softening. However, careful tuning of the focus via adjustment of the numerical aperture (NA), combined with appropriate laser parameters (laser pulse energy) can confine the free electron density to around 1021 cm-3. In this regime, the thermal effects are absent and local modification of biological media has exclusively chemical origins. The operating envelope is bounded above by increased thermal effects, which denature ECM and soften the tissue, and bounded below by insufficient chemical alteration, leading to negligible enhancement of properties.

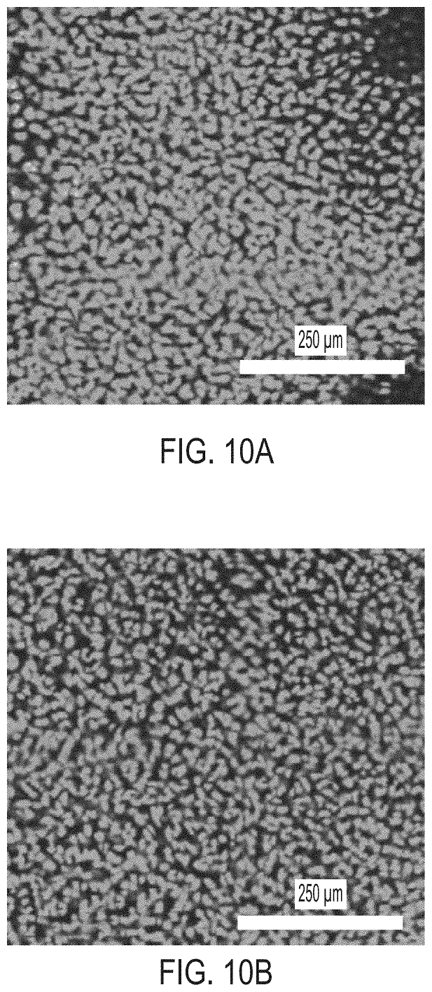

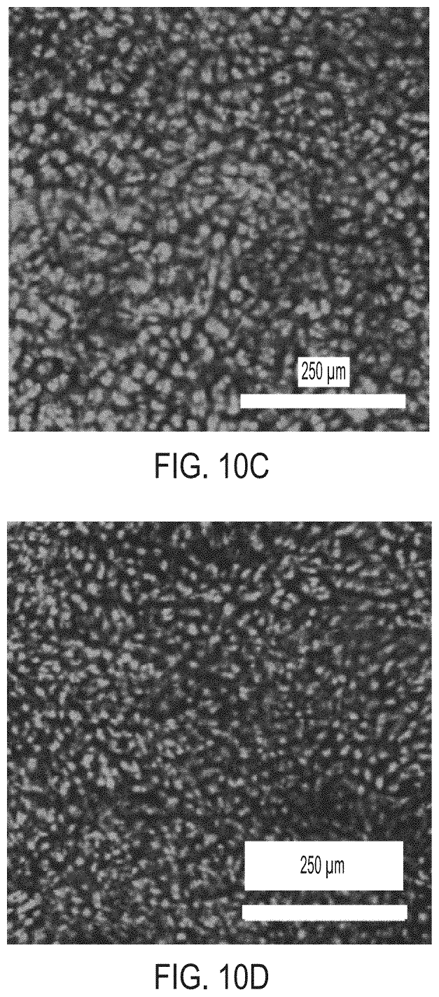

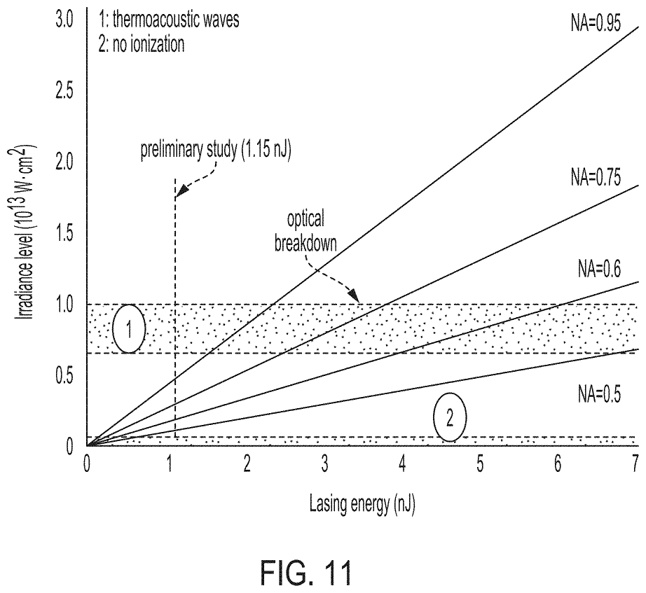

FIGS. 10A and 10B show live/dead staining of immature bovine cartilage explants (PS3). FIG. 10A shows control and, FIG. 10B, laser-treated, at 24 h; (FIG. 10C) control and (FIG. 10D) laser-treated at 2 wks.

Immature bovine tissue is used in tests because of its plentiful supply under relatively controlled conditions of age and health, a benefit to both devitalized explants and live; in particular, its elevated metabolic response, relative to mature cartilage, makes it well suited for exploring the downstream, long-term effects of laser treatment in vitro using explant cultures that last a few weeks only. Inducing OA-like damage to these bovine explants provides a higher bar for testing the effectiveness of laser treatment. Human cartilage from male and female OA joints is most directly relevant to the clinically translational aims of this application. Studies have shown that human cartilage from OA joints exhibit significantly lower Young's modulus than cartilage from healthier joints, even when the tissue is visually normal or just fibrillated. Thus, this tissue represents the prime target for laser treatment. To induce OA-like degeneration in bovine cartilage, the samples may be worn via mechanical means. The effectiveness of the laser treatment may be primarily assessed via quantitative characterization of mechanical properties, including the equilibrium compressive Young's modulus and wear resistance. Secondary measures of damage and repair will include standard measurements of biochemical composition (water, collagen and proteoglycan content), histology, and semi-quantitative microscopic imaging such as polarized light microscopy (PLM) and two-photon fluorescence (TPF) imaging. Direct quantitative measurement of Crosslinks produced by laser treatment is not currently possible, as the nature of all the Crosslinks has not been fully identified.

FIG. 11 shows operating range for laser parameters including a theoretical envelope of effective and safe laser energy domain between the shaded zones.

Due to its avascular nature, the metabolic requirements of adult cartilage are mostly serviced by diffusion from synovial fluid. Oxygen levels in the tissue are rather low, inhibiting glycolysis and ECM production. In this environment, ROS play significant physiological roles, including cell activation, proliferation and matrix modeling. Sustained levels of ROS are required for the maintenance of ion homeostasis in chondrocytes. However, in response to partial oxygen pressure variations, mechanical loading and inflammatory mediators, chondrocytes produce elevated levels of ROS. These are mainly nitric oxide (*NO) and superoxide anion (O2-), which are then responsible for generation of other radicals such as peroxynitrite (ONOO—) and hydrogen peroxide (H2O2). Cellular response to ROS is a function of its redox status, and as long as the reducing capacity of the cell matches its oxidant level, ROS remain within their physiological concentrations. When ROS production exceeds the antioxidizing potential of the cell, an ‘oxidative stress’ is generated which contributes to cartilage degradation. Imbalance between catabolic and anabolic activity results in oxidation of membrane phospholipids, nucleic acids, and other intracellular and extracellular components, eventually leading to cell death and release of cellular content into the extracellular environment. The overproduction of ROS in pathological cartilage has been associated with the accumulation of lipid peroxidation products and nitrotyrosine in situ. This well-documented role of ROS in OA-afflicted articular cartilage requires careful consideration of potentially adverse effects of laser-induced ROS. Laser treatment induces short-lived bursts of non-nitric ROS, primarily from ionization of the cartilage interstitial water, and these ROS then mostly interact with collagen in the ECM.

Specifically, once the laser treatment operating envelope has been established the scope of the optimization may be narrowed as indicated above where the objective is to identify the range within that envelope that maintains cell viability and tissue health both short-term (24 h post laser treatment) and long-term (after 4-week in vitro culture with daily dynamic loading), while confirming the maintenance of improved mechanical properties.

In addition to qualitative assessment of ROS production, it may determine whether the treatment has induced oxidative stress by measuring levels of F2-Isoprostanes. Isoprostanes, prostaglandin-like compounds, are formed by ROS-assisted peroxidation of arachidonic acid. When acute or chronic joint damage is present, arachidonic acid is the primary fatty acid produced by the metabolic conversion of cell membrane phospholipids. F2-Isoprostanes are seen as reliable indicators of oxidative stress due to their stability and clear increase in concentration when compared against quantities detectable in normal tissues.

Described tests treat a 03 mm region in 5 min. A practical tool for in situ laser treatment may employ a fiber optic-based probe to apply the laser light arthroscopically. The arthroscopic probe may be developed such that it operates within the range identified above. Preferably, clinical and engineering considerations of such a probe may constrain the laser procedure to last no longer than common cartilage debridement (e.g., 30 minutes or less). To meet this constraint, the focusing tip of the probe may be designed such that the required energy density is deployed at NA values that accommodate a relatively large focal volume. Since the focal volume has the shape of a Gaussian ellipsoid with both radii being a strong function of NA, relatively loose focusing will result in a large depth of field. This favorable characteristic increases the penetration depth. The laser intensity within the focal volume may follow a Gaussian distribution with the peak limited to prevent it exceeding an upper threshold at the center of the ellipsoid.

Experiments may identify the envelope of effective and safe laser operating parameters (laser pulse energy and numerical aperture NA) using devitalized healthy immature bovine cartilage (experiment E1a), devitalized OA-like immature bovine cartilage (E1b) and mature human cartilage from OA joints (E1a). OA-like conditions for immature bovine cartilage may be created by mechanically wearing the articular surface. In all experiments, lased samples may be compared to unlased controls that have been subjected to sham treatment conditions (i.e., placed in the same bath as lased samples, but away from the laser beam). Lased and unlased cartilage explants may be harvested from adjoining sites and serve as pairs in a repeated measures design. Experiment may identify the widest range of laser energy and NA that produces effective enhancement of the equilibrium compressive modulus Young's modulus (measured as described in the present disclosure) and the wear resistance WR (described in the present disclosure). The effectiveness of laser treatment may be assessed by statistically comparing these measures in paired comparisons. For example, if Young's modulus-lased is found to be statistically greater|equal|less than Young's modulus-unlased for a given set of laser parameters (laser energy, NA), it may be concluded that these parameters are effective|ineffective|unsafe with respect to this measure; similar assessments may be performed for WR. The final assessment for each (laser energy, NA) combination may be based on the worst outcome between Young's modulus and WR. Upon completing E1a, this protocol may be repeated with E1b and E1c, each time using only the range of (laser energy, NA) parameters found effective in the preceding experiment. This approach eliminates extra testing in ranges already deemed ineffective or unsafe.

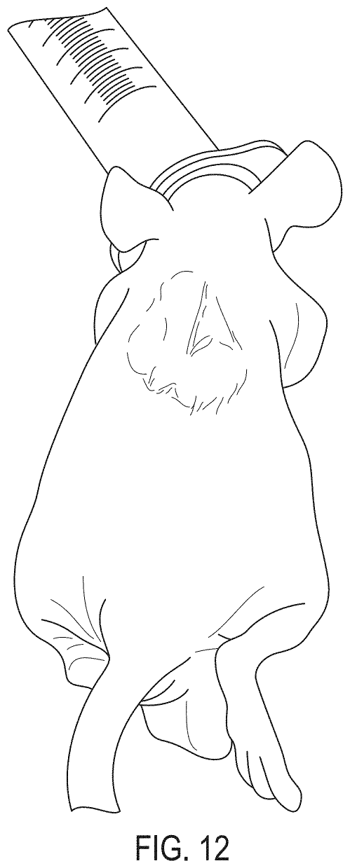

FIG. 12 shows subcutaneous dorsum pouch of an athymic mouse with 4 implanted engineered cartilage constructs (06 mm×1.4 mm). Cartilage explants may be used in SA3. FIG. 12 shows a pouch extending from the back of the nude mouse toward the viewer.

The choice of NA determined by the objective may be optimized. A lower threshold of an operating envelope may be established in tests by examining treating solution of spin-trap reagent 5,5-dimethy-1-pyrroline-N-oxide solved in Dulbecco's phosphate-buffered saline. Upon laser irradiation, electron paramagnetic resonance spectroscopy (described in the present disclosure) may be used to determine whether ionization is sufficient to produce ROS. Starting with a lowest NA (e.g., 0.5), the laser pulse energy laser energy may be one of (0.5, 2.5, 3.5, 4.5, 5.5) nJ, with the latter being the theoretical energy at which thermoacoustic waves start to occur. Laser energy may be increased by 1 nJ increments. The upper threshold of the operating envelope is bound by the amount of thermal accumulation, induced by plasma-initiated thermoacoustic waves, which is assumed to start approximately at 0.75 of the optical breakdown threshold. The statistical design for E1 is described herein. Depth-dependent measurements may be performed of Young's modulus as described herein, standard measurements of tissue composition and histology (as described in the present disclosure), and light microscopy (PLM and TPF), to achieve a more thorough understanding of the laser treatment effects.

The range of operating parameters (laser energy, NA) may be narrowed by testing (a) short-term and (b) long-term viability of lased live healthy immature bovine cartilage (experiment E2) and adult human cartilage from OA joints (experiment E3) against unlased controls, using in vitro culture up to 4 weeks. Laser treatment may be performed under sterile conditions. In short-term experiments (E2a and E3a), viability may be assessed 24 h after laser treatment as disclosed herein. In long-term experiments (E2b and E3b), live explants may be returned into culture for four weeks, as described elsewhere herein, prior to assessing cell viability and tissue health. The full range of mechanical, biochemical, histological and imaging assessments described in SA1 will also be performed in SA2. The primary outcome measure for SA2 is cell viability: The range of effective (laser energy, NA) operating parameters may be narrowed by first identifying those pairs that maintain an acceptable level of viability compared to the control treatment: Based on common criteria for storage of live osteochondral allografts, a threshold of 75% viability may be used as a measure of effectiveness. The range of effective operating parameters may be further narrowed based on comparing Young's modulus and WR between lased and unlased explants.

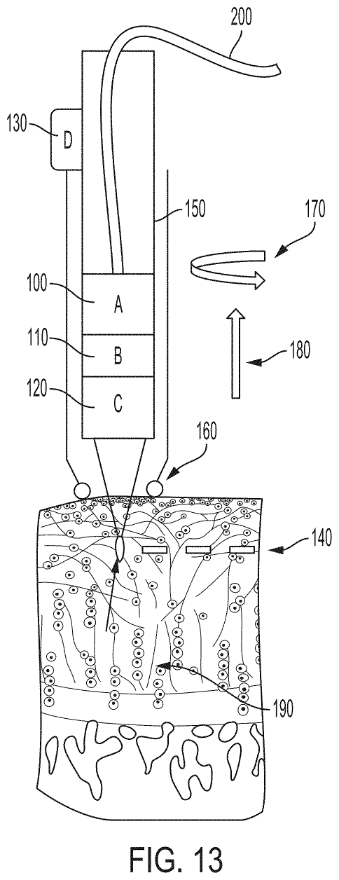

FIG. 13 shows a schematic diagram of the fiber optic probe and illustrates its application to a tissue within a focal volume 190. Focusing optics are shown with a fiber-to-collimator adaptor 100. A fiber collimation package is indicated at 110. A high-NA objective is indicated at 120 and a micrometer actuation system is indicated at 130. A twist handle 150 is used to rotate as illustrated at 170 and to advance/retract as illustrated at 180. A spacer 160 adjusts by desired increments and change the laser treatment plane as shown at 140. The fiber option cable is indicated at 160. A fiber optic cable is indicated at 200.

Toxicity and in vivo assessment of laser treated and control live human cartilage may be performed via implantation into subcutaneous pouches on the dorsum of athymic nude mice (Charles River, strain code 490). This is a standardized and accepted animal model for determining biocompatibility, initial safety, and implant viability. For this experiment E4, four cartilage explants were implanted in each mouse (2 control-lased pairs, one from male and the other from female human OA cartilage donors). Following 4 and 8 weeks of implantation, harvested explants may be evaluated for viability, mechanical and biochemical properties and histologic characteristics (similar to experiment E3), as well as microbial culture of the implants and full necropsy of the mouse to examine liver, kidneys, bone marrow and brain tissues for evidence of toxicity. A total of 14 nude mice may be used (7 male and 7 female) to study in vivo response of implanted cartilage (total of 56 live cartilage explants) at two time points, 4 and 8 weeks. An 8 week maximum time course is chosen as cartilage loses properties in the subdorsum pouch, presumably due to the lack of mechanical loading to provide nutrients to tissues, as noted in prior studies and reports in the literature.

A fiber optic probe may be modeled based on systems for fiber optic fluorescence imaging and spectroscopy. The surgeon inserts a probe during arthroscopy and treats the region of interest by moving the probe over the articular surface. The spacer gently touches the cartilage to ensure that the focal volume remains approximately at a prescribed depth. After a ‘treatment plane’ has been laser irradiated, the physician adjusts the spacer by finite increments (e.g., 50-100 μm) with a convenient half-turn of the micrometer-like handle, so that the focal volume sweeps at a different depth.

A preliminary prototype was constructed and evaluated, showing that a fiber optic based probe can deliver average power and beam profile similar to free space optic system. This initial prototype has been realized with single mode, ‘conventional’ fiber-optic cable. The next generation prototype will incorporate hollow core photonic crystal fiber, specifically designed to transmit 1060 nm laser wavelength. This class of fiber will circumvent issues that arise with fiber-based laser systems, such as group velocity dispersion and self-phase modulation, which may distort the pulse's temporal profile, having a detrimental effect onto the processing conditions. The optical fiber may be coupled onto a custom-built pen-like tip, which will contain optical objectives. Custom, high-NA objective lenses with NA ranging from 0.5 to 0.95 may be fabricated via injection molding by following, then mounted onto the tip. Probe embodiments may be mounted on a motorized stage to replicate results with a free-space optics system and may incorporate a load sensor to provide visual LED feedback if operator applies too much pressure.

Testing of probes may use adult human OA cartilage under conditions developed for experiments described. New results may be compared against findings in E3, and further optimized via alteration of focusing (NA) and laser (laser energy) parameters. Once processing parameters for this fiber optic based system are established, further testing may be performed on human OA cadaver joints, in a simulated arthroscopic procedure. Only OA-afflicted knee joints may be used. Following arthroscopic treatment of OA joints with the probe, explants may be obtained from treated regions and neighboring sites and analyzed as described in experiments.

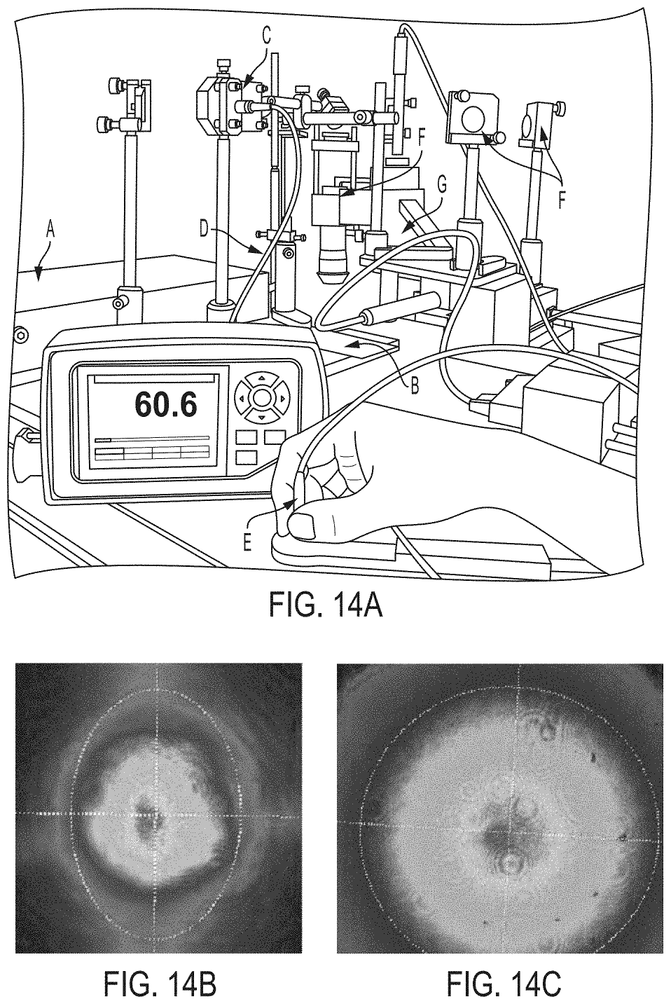

FIG. 14A shows laser setups for fiber optic probe & free-space optics. A. Femtosecond laser; B. Tissue sample area; C. 3-axis translation mount; D. Fiber optic cable; E. Probe tip; F. Focusing optics; G. 3-axis motorized actuator. Gaussian laser beam profile with ˜60 mW power is obtained from a free-space objective (FIG. 14B) and a fiber-based probe (FIG. 14C).

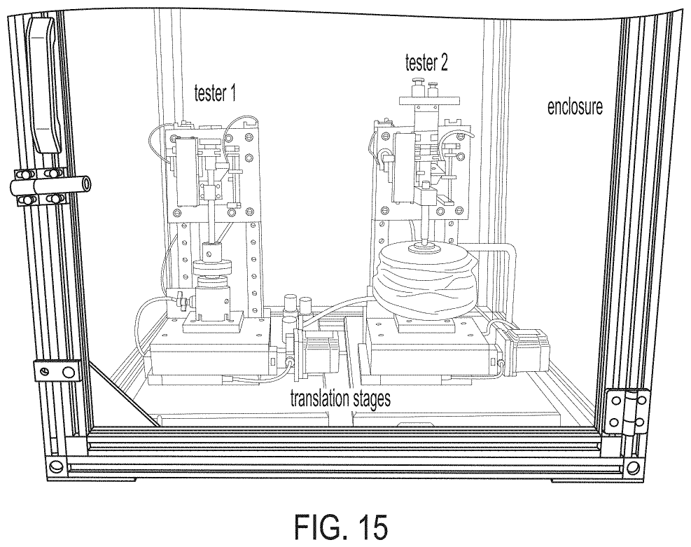

FIG. 15 shows two friction testing devices.

Immature bovine explants may be cultured in chemically defined media designed to maintain homeostatic conditions (no changes in mechanical and biochemical properties), and subjected to daily dynamic compressive loading (±2% strain amplitude at 0.5 Hz, over 10% tare strain, 30 min/day) as described in prior work by applicants, explant and tissue engineering studies. chemically defined media is a modification of typical cartilage culture media tuned by adjusting levels of glucose, insulin, hydrocortisone, and amino acids to physiologically relevant concentrations: Dulbecco's modified Eagle's medium with a modification for 0.25× amino acids, 1 mg/mL glucose, 1 mg/ml bovine serum albumin, insulin-transferrin-selenous acid (0.0003×ITS+Premix, Corning), 1% antibiotic-antimycotic, 0.03 μg/ml hydrocortisone, 110 μg/ml sodium pyruvate, and 12 μg/ml ascorbate-2-phosphate. Human OA explants may be cultured in chondrogenic media (CM) as described in previous studies: high glucose (4.5 mg/mL) Dulbecco's modified Eagle's medium containing 100 nM dexamethasone, 110 mg/mL sodium pyruvate, 50 mg/mL 1-proline, 1% ITS+Premix (final medium concentrations: 6.25 mg/mL human recombinant insulin, 6.25 ng/mL selenous acid, 6.25 mg/mL human holotransferrin, 1.25 mg/mL bovine serum albumin, and 5.35 mg/mL linoleic acid), 1% antibiotic-antimycotic, 50 μg/mL ascorbate-2-phosphate.

Examples of femtosecond laser oscillators that may be employed are: (1) Chameleon Ultra II (Coherent, Inc.) that delivers 140 fs long laser pulses at 80 MHz repetition rate, with tunable wavelength (680 nm-1080 nm), and (2) High-Q (High-Q Laser, Austria) with temporal pulse width of 99 fs and 52 MHz repetition rate, with output wavelength centered around 1060 nm. Laser (1) may be used for treatment in all experiments. Either laser may be coupled with a 3-axis translational stage (Thorlabs, Inc.) configured to deliver laser pulses through a high numerical aperture objective.

Two custom, two-axis loading devices with 6-degree-of-freedom load cells (JR3 Inc. #20E12A4) are available, within a clean environment. These devices include a translation stage (JMAR xy stage) with linear encoder (RSF Electronics MSA 65x series, 5-10 μm resolution), which may be stably operated at speeds up to 10 mm/s. Custom LabVIEW software (National Instruments Corporation #LabVIEW 2010) and an associated motion controller (National Instruments Corporation #7354) provide continuous measures of specimen thickness, position, applied loads, and reaction loads. The wear testing protocol against a glass slide is based on previously published studies: Constant load (contact stress=0.18 MPa) and reciprocal sliding (±5 mm at 1 mm/s) for 4 h (N=1,440 cycles). The wear resistance ratio WR is the ratio of Young's modulus prior and subsequent to wear testing on a sample.

Early damage to the ultrastructure of the collagen extracellular matrix may cause swelling of the tissue due to the Donnan osmotic pressure of charged proteoglycans. A swollen matrix exhibits a lower fixed-charge density, which reduces the equilibrium compressive modulus Young's modulus of damaged cartilage, as found in inventor's recent study of cartilage damage against orthopaedic implant materials. To measure Young's modulus across the full thickness of an explant, the devices may be used and protocols for unconfined compression stress-relaxation described in inventor's previous studies. Furthermore, to distinguish properties in the laser-treated zone (˜300 μm from articular surface) from cartilage in the deeper zone, to perform depth-dependent measurements of Young's modulus using a microscope-mounted testing device coupled with digital image correlations as described in inventor's previous studies.

Cell viability may be assessed with live/dead staining and changes in cell proliferation and metabolism (DNA & MTT assay). For a subset of samples, cell counting may be performed by first digesting explants with collagenase (Sigma-Aldrich), filtering the cartilage digest through a 70 μm mesh (McMaster-Carr) to remove debris, and analyzing it with a particle sizing and counting analyzer (Multisizer 4, Beckman Coulter). For biochemistry, samples may be weighed wet, lyophilized, weighed dry, then digested in 0.5 mg/mL proteinase K solution (Fisher Scientific) overnight at 56° C. Water content may be determined from the dry and wet weights. Sulfated GAG content may be determined by dimethylmethylene blue spectrophotometric assay. Collagen content may be assessed by orthohydroxyproline assay of acid-hydrolysates of proteinase K digests assuming a 1:7.64 OHP-to-collagen mass ratio. Biochemical contents may be normalized to tissue sample pre-test weights. Samples for histology may be fixed, sectioned, and stained with Safranin-O for charged proteoglycans, and with Picrosirius red for collagen. Collagen-specific immunohistochemistry (type I for fibrous tissue formation, II for hyaline maintenance, and X for hypertrophy) may be performed as described in inventor's previous studies.

Post treatment presence of ROS in live explants may be assessed with human femoral condyle spectrometer (Bruker BioSpin EMX Electron Paramagnetic Resonance Spectrometer, Bruker BioSpin GmbH, Germany). Sample preparation for human femoral condyle spectroscopy may be performed by the following: explants may be digested overnight via enzyme solution, and single suspension cells placed into spin trapping reaction mixture consisting of 4-pyridyl 1-oxide-N-tert-butylnitrone (4-POBN) (10 mM), ethanol (170 mM), Diethylenetriaminepentaacetic acid (DTPA) (0.1 mM), phorbol 12-myristate 13-acetate (PMA) (100 ng·ml), with the final volume of the mixture being 0.2-0.5 ml due to addition of phenol-free Hank's balanced salt solution (HBSS). The reaction mixture may be placed into a quartz capillary tube and transported to human femoral condyle spectrometer. Commercially available immunoassay kits may be utilized to assess levels of F2-isoprostanes in cartilage.

Cartilage samples for PLM may be sectioned without fixation and imaged, for example, on an Olympus BX60 microscope, with a drop-in U-POT polarizer and Olympus DP72 camera. PLM may be used to detect changes in the thickness of the superficial, middle and deep zones of an articular layer following wear testing. Relative crosslink density may be evaluated by semi-quantitative analysis of images obtained with TPF microscopy. TPF imaging may be performed with a microscope (Bruker) equipped with a tunable lase such as a Mai Tai Deep See Ti:Sapphire laser (Spectra Physics) as excitation source. A 10×/0.6 NA water immersion objective (e.g., Olympus) may be used to collect fluorescence signals which may be registered with two different photomultiplier tubes, one in red (580-620 nm) and the other in green (480-570 nm) wavelength regime. The excitation wavelength may be set to 826 nm for collagen.

Live explants may receive ascorbic acid during culture. The mice diet may be supplemented with ascorbic acid. Levels of radicals immediately after laser treatment may be assessed via human femoral condyle.

The approach and methods may also be applied by:
1) Using laser crosslinking to “glue” collagenous tissues together across an interface, by creating crosslinks between collagen molecules/fibrils/fibers located on both sides of the interface.
2) Adding a collagen-based liquid or gel at the interface between collagenous tissues and using laser crosslinking to increase crosslinking sites across the interface, thus enhancing the “gluing” mechanism.

This technology can have widespread applications in the treatment of to: (a) repairing meniscal tears in the tibiofemoral and temporomandibular joints; (b) gluing cartilage surfaces in osteochondral repairs that use allografts or autografts (e.g., “mosaicplasty”); (c) repairing ligament tears; (d) repairing tendon tears; (e) repairing skin cuts and tears as may result from surgery including plastic surgery.

According to first embodiments, the disclosed subject matter includes a method of stiffening cartilage. The method includes exciting reactive oxygen species by creating a multiple-photon excitation in a cartilage, the power, photon energy, and duration of the exciting being limited to prevent heating or optical breakdown in the cartilage.

In variations thereof, the first embodiments include ones in which the cartilage exhibits osteoarthritis. In variations thereof, the first embodiments include ones in which the exciting includes scanning a laser on a surface of the cartilage. In variations thereof, the first embodiments include ones in which the laser is delivered as pulses carrying nano-joule (nJ) energy. In variations thereof, the first embodiments include ones in which the laser is not focused in the cartilage so as to excite a volume within the cartilage. In variations thereof, the first embodiments include ones in which each portion of the surface is scanned not more than twice.

In variations thereof, the first embodiments include ones in which each portion of the surface is scanned less than twice. In variations thereof, the first embodiments include ones in which each portion of the surface is scanned no more than once.

According to second embodiments, the disclosed subject matter includes a method of repairing tissues. The method includes exciting reactive oxygen species by creating a multiple-photon excitation in tissue media including cartilage, ligaments, tendon tissue, skin, and connective tissues generally, to repair or improve damage to the tissue media that forms an interface where the tissue media is divided. The power, photon energy, and a duration of the exciting is limited to prevent heating or optical breakdown in the tissue media.

In variations thereof, the second embodiments include ones that include applying a collagen-containing liquid at said interface. In variations thereof, the second embodiments include ones in which said tissue media includes cartilage. In variations thereof, the second embodiments include ones in which the exciting is performed across said interface. In variations thereof, the second embodiments include ones in which the exciting is effective to create crosslinks between collagen molecules/fibrils/fibers located on all surface and sides of the interface. In variations thereof, the second embodiments include ones in which the exciting includes scanning a laser on a surface of the tissue media. In variations thereof, the second embodiments include ones in which the laser is delivered as pulses carrying nano-joule (nJ) energy. In variations thereof, the second embodiments include ones in which the laser is not focused in the tissue media so as to excite a volume within the tissue media. In variations thereof, the second embodiments include ones in which each portion of the surface is scanned not more than twice.

In variations thereof, the second embodiments include ones in which each portion of the surface is scanned less than twice. In variations thereof, the second embodiments include ones in which each portion of the surface is scanned no more than once.

The foregoing descriptions apply, in some cases, to examples generated in a laboratory, but these examples can be extended to production techniques. For example, where quantities and techniques apply to the laboratory examples, they should not be understood as limiting.

Features of the disclosed embodiments may be combined, rearranged, omitted, etc., within the scope of the invention to produce additional embodiments. Furthermore, certain features may sometimes be used to advantage without a corresponding use of other features. It is, thus, apparent that there is provided, in accordance with the present disclosure, treatments and procedures for collagen containing tissues. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicants intend to embrace all such alternatives, modifications, equivalents, and variations that are within the spirit and scope of the present invention.

What is claimed is:

1. A method of stiffening cartilage, comprising:
exciting reactive oxygen species by creating a multiple-photon excitation in a cartilage, the power, photon energy, and duration of the exciting being limited to prevent heating or optical breakdown in the cartilage.

2. The method of claim 1, wherein the cartilage exhibits osteoarthritis.

3. The method of claim 1, wherein the exciting includes scanning a laser on a surface of the cartilage.

4. The method of claim 3, wherein the laser is delivered as pulses carrying nano-joule (nJ) energy.

5. The method of claim 4, wherein the laser focus is such that a volume within the cartilage is excited.

6. The method of claim 4, wherein the exciting includes scanning a laser on a surface of the cartilage.

7. The method of claim 3, wherein each portion of the surface is scanned not more than twice.

8. The method of claim 3, wherein each portion of the surface is scanned no more than once.

9. A method of repairing tissues, comprising:
exciting reactive oxygen species by creating a multiple-photon excitation in tissue media including cartilage, ligaments, tendon tissue, skin, and connective tissues generally, to repair or improve damage to the tissue media that forms an interface where the tissue media is divided,
wherein power, photon energy, and a duration of the exciting is limited to prevent heating or optical breakdown in the tissue media.

10. The method of claim 9, further comprising applying a collagen-containing liquid at said interface.

11. The method of claim 9, wherein said tissue media includes cartilage.

12. The method of claim 9, wherein the exciting is performed across said interface.

13. The method of claim 9, wherein the exciting is effective to create crosslinks between collagen molecules/fibrils/fibers located on all surface and sides of the interface.

14. The method of claim 9, wherein the exciting includes scanning a laser on a surface of the tissue media.

15. The method of claim 14, wherein the laser is delivered as pulses carrying nano-joule (nJ) energy.

16. The method of claim 15, wherein the laser focus is such that a volume within the tissue media is excited.

17. The method of claim 15, wherein the exciting includes scanning a laser on a surface of the tissue media.

18. The method of claim 14, wherein each portion of the surface is scanned not more than twice.

19. The method of claim 14, wherein each portion of the surface is scanned no more than once.
